(19) United States
(12) Patent Application Publication
(10) Pub. No.: US 2011/0091577 A1
(43) Pub. Date: Apr. 21, 2011

(54) DRUG RELEASE COATINGS ON CALCUIM PHOSPHATE AND USES THEREOF

(76) Inventor: Lance D. Silverman, New York, NY (US)

(21) Appl. No.: 12/736,605

(22) PCT Filed: Apr. 15, 2009
(86) PCT No.: PCT/US09/02356

§ 371 Date: Dec. 1, 2010

Related U.S. Application Data
(60) Provisional application No. 61/125,626, filed on Apr. 25, 2008.

(51) Int. Cl. A61K 33/24; A61K 47/02; A61K 47/42; A61K 31/7028; A61K 31/7036; A61K 31/7042; A61P 31/00; A61P 29/00; A61P 23/00

(52) U.S. Cl. 424/649; 514/769; 514/23; 514/40; 514/29

(57) ABSTRACT

The invention provides implantable drug releasing materials comprising a calcium phosphate composition, a biodegradable polymer adsorbed onto the calcium phosphate composition, wherein the polymer comprises acidic amino acid residues, and a drug adsorbed onto or reacted with the polymer. The invention is further directed to dental and bone implants and implantable medical devices comprising the implantable drug releasing material, methods for preparing the implantable drug releasing material, and methods for delivering the implantable drug releasing material to bone or teeth.

### CROSS-REFERENCE TO RELATED APPLICATION

This application claims the benefit of U.S. Provisional Patent Application No. 61/125,626, filed on Apr. 25, 2008, the content of which is hereby incorporated by reference into the subject application.

### FIELD OF THE INVENTION

The present invention generally relates to implantable drug releasing materials comprising a calcium phosphate composition, a biodegradable polymer adsorbed onto the calcium phosphate composition, where the polymer comprises acidic amino acid residues, and a drug adsorbed onto or chemically bound to the polymer; methods of preparing the materials; and use of the materials in particular as bone and dental implants and with implantable medical devices.

### BACKGROUND OF THE INVENTION

Throughout this application various publications are referred to in parenthesis. Citations for these references may be found at the end of the specification immediately preceding the claims. The disclosures of these publications are hereby incorporated by reference in their entireties into the subject application to more fully describe the art to which the subject application pertains.

The need to substitute, reconstruct, or regenerate damaged or lost bone tissue in the human body while effectively delivering a drug to the desired site is a serious challenge posed to the medical community. Implantable drug delivery devices are known in the art and a number are commercially available. These drug delivery devices are composed of a variety of biomaterials, such as metals, ceramics, polymers, and glass.

However, these devices are not without their shortcomings. For example, bone cements used to fix metallic prostheses such as an artificial hip, typically based on polymethymethacrylate, are commercially available preloaded with an antibiotic. However, the incorporation of the antibiotic can weaken the bone cement, thereby interfering with its primary function. Additionally, most of the incorporated drug is never released, but rather remains trapped in the polymer. In other available devices, the materials composing these devices are not bioresorbable and therefore remain in the body or need to be removed in a second surgery. For example, antibiotic-loaded polymethylmethacrylate beads on a wire must be removed in a second procedure, incurring additional risk, pain and expense.

Drug coatings on implants can also interfere with the implants' primary function to promote bone replacement and can have other adverse effects. For example, polylactide and polyglycolide polymers or copolymers are used as drug release materials, including coatings (Schmidmaier et al., 2006a,b). However, these materials can hydrolyze to produce acidic products (Agrawal et al., 1997) that can degrade drugs and can shed particles that can cause an inflammatory response (Cordewener et al., 2000; Hovis et al., 1997).

Bioresorbable controlled drug release devices that do not remain in the body are also available, for example, collagen sponges loaded with drugs. However, in the case of an orthopedic implant, these would need to be placed outside of the site where the growth of bone tissue is desired, since the collagen sponge does not serve as a bone substitute. This limits the effectiveness of the release device since the drug would need to diffuse from the sponge into the bone graft itself. An antibiotic preloaded bone graft material is available from Wright Medical Technologies, where the graft material is designed to be replaced by natural bone following surgery and the residual material resorbed. However, this bone graft material, based on calcium sulfate hemihydrate (plaster of Paris), is not popular because the graft material is resorbed faster than new bone can be formed, leaving a gap in the bone.

Therefore, there is a compelling need to develop improved drug-release coatings for orthopaedic implants for the local delivery of lifesaving medicines, such as antibiotics or chemotherapeutic agents, that do not exhibit the shortcomings of the drug-release coatings and materials currently available.

### SUMMARY OF THE INVENTION

The present invention is directed to implantable drug releasing materials comprising (a) a calcium phosphate composition, (b) a biodegradable polymer adsorbed onto the calcium phosphate composition, wherein the polymer comprises acidic amino acid residues, and (c) a drug adsorbed onto or chemically bound to the polymer. The present invention is further directed to dental or bone implants comprising the implantable drug releasing material. The present invention is further directed to methods for preparing implantable drug releasing materials, the implantable drug releasing material formed by these methods, and methods for delivering the implantable drug releasing material to bone or teeth.

### BRIEF DESCRIPTION OF THE DRAWINGS

FIG. 1A-1C. Models of drugs bound to implant surfaces via absorbed polypeptides. FIG. 1A shows an acidic, linear polypeptide with “loops” and “tails” that stick out from the surface and bind drug molecules. FIG. 1B shows a block copolypeptide that has one polypeptide block optimized for surface adsorption and the second polypeptide block optimized for binding the drug. FIG. 1C shows a branched polypeptide, where the many branches prevent the polymer from adsorbing flatly on the surface by steric interference, so that segments stick out into solution where they can bind drug molecules.

FIG. 2A-2D. Gentamicin and vancomycin controlled release results. FIG. 2A shows the release profile of gentamicin from gentamicin/polyglutamate-hydroxyapatite (G/pgHA) and gentamicin/hydroxyapatite (G/HA) control. FIGS. 2B-2C show the release profile of gentamicin from G/pgHA and G/HA control (2B) and integrated release (2C) when the buffer is periodically removed and replaced from each sample. Same symbols apply in 2A-2C for G/pgHA and G/HA. FIG. 2D shows the integrated release profile of vancomycin from vancomycin/polyglutamate-hydroxyapatite (V/pgHA) and vancomycin/hydroxyapatite (V/HA) control when the buffer is periodically removed and replaced from each sample.

### DETAILED DESCRIPTION OF THE INVENTION

The present invention provides an implantable drug releasing material comprising a) a calcium phosphate composition, b) a biodegradable polymer adsorbed onto the calcium phosphate composition, wherein the polymer comprises acidic amino acid residues, and c) a drug adsorbed onto or chemically bound to the polymer.

The invention also provides a method of preparing an implantable drug releasing material comprising a) adsorbing a biodegradable polymer to a calcium phosphate composition, wherein the polymer comprises acidic amino acid residues; and b) adsorbing or chemically binding a drug onto the polymer. The calcium phosphate composition, for example, can form part of an implantable medical device or implant, or can be coated onto an implantable medical device or implant.

Preferably, the calcium phosphate composition comprises hydroxyapatite or tricalcium phosphate.

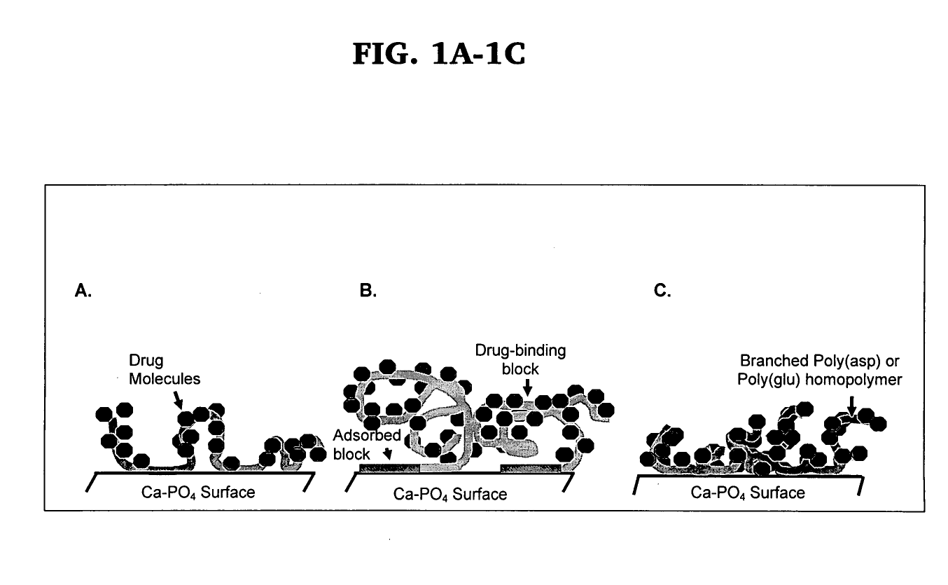

Preferably, the polymer is a polypeptide polymer (e.g., FIG. 1A). Preferably, the polymer comprises residues of aspartic acid and/or glutamic acid. Preferably, the polymer comprises phosphoserine. The polymer can be, for example, a poly(glutamic acid) polymer or a poly(aspartic acid) polymer. The polymer can comprise branched polypeptides (e.g., FIG. 1C). The polymer can be a block co-polymer. As used herein, a “co-polymer” is a polymer derived from two monomeric species, as opposed to a homopolymer where only one monomer is used. The monomers in a co-polymer can occur in long alternate sequences or blocks. A “block co-polymer” means a polymer comprising two or more chemically different segments, or blocks, connected by a covalent linkage. The block co-polymer can comprise one block comprising peptide sequences with acidic residues and another block optimized to bind a drug (e.g., FIG. 1B). The polymer can be formed as a monolayer. The polymer can be bound to the calcium phosphate composition, for example, by ionic interaction.

The polymers used in the present invention preferably fulfill several criteria. They adsorb strongly to calcium phosphate mineral through acidic peptide sequences (Tsortos and Nancollas, 1999). They are designed to bind to and later release specific drugs. They are biocompatible and biodegradable. The polymers are biomimetic, i.e., they mimic many attributes of naturally occurring proteins that control mineral formation in bones and teeth. The proteins that control biomineralization, such as bone sialoprotein and osteopontin, adsorb strongly to calcium phosphate (hydroxyapatite) through acidic peptide sequences that are rich in aspartate, glutamate, and phosphoserine amino acid residues (Goldberg et al., 2001; Tsortos and Nancollas, 2002). They can also perform secondary functions, such as cell signaling or attaching mineral to other materials (Qin et al., 2004). They must be structured such that protein segments that perform secondary functions do not interfere with the protein's capacity to bind to mineral, for example through steric interference.

Block co-polymers have the capability to provide a biomaterial having different polymer segments optimized for different functions, and the capability to display a broad range of amphiphilic characteristics (Jo et al., 2006; Vakil et al., 2006). The most frequently used route to synthesize block copolymers that contain polypeptide blocks is the ring-opening polymerization of protected amino acid-N-carboxyanhydrides (NCA) (Deming, 1997). Variations on this synthetic approach can be used to make the block co-polymers and branched polypeptides of the present invention.

Many types of drugs can be beneficially used in connection with the drug releasing material of the subject invention. These include, but are not limited to, antibiotics, chemotherapeutic drugs, analgesics, growth factors, anesthetics, anti-inflammatory drugs and cell signaling compounds. Examples of acceptable antibiotics are known in the art, and include without limitation, aminoglycosides (including gentamicin and tobramycin) and vancomycin.

One embodiment of the present invention pertains to the use of antibiotics for the prevention of infection following surgery (e.g., osteomyelitis). Examples of antibiotics that are commonly used in orthopaedic applications include, but are not limited to, gentamicin, tobramycin, and vancomycin. Results described below show that clinically significant amounts of the antibiotic gentamicin can be loaded onto and released from the materials of the invention. Importantly, controlled release materials that are commercially available or described in the literature use concentrations of antibiotics that could kill osteoblasts and thus interfere with tissue scaffold-type implants. However, lower concentrations may act prophylactically to prevent infection while not interfering with bone regeneration, as discussed in a recent publication (Silverman et al. 2007). One potential benefit of this approach is that the implants may prevent infection, but release all of the antibiotic within two to four weeks to avoid breeding antibiotic-resistant bacteria. After about two weeks, new vasculature invades the surgical site and can carry in the body's natural defenses or systemically administered drugs.

Chemotherapeutic drugs that can be used in the present invention include, but are not limited to, cisplatin. Cisplatin can be bound to aspartate or glutamate carboxylic acid groups through ligand substitution at platinum (Nishiyama et al., 1999). The bonding involves a coordinate bond.

In one embodiment of the invention, the ratio of the number of monomers in the polymer to the number of drug molecules is about 5:1 to about 20:1, and preferably about 10:1.

The drug can be bound to the polymer, for example, by ionic interaction or by a coordinate bond with a carboxyl group or other covalent bond. An example of ionic interaction is the incorporation of charged drug molecules by their ionic attraction to mineral-adsorbed polymers of opposite charge.

The present invention is also directed to an implant (e.g., a dental implant or a bone implant) comprising any of the implantable drug releasing materials described herein. Bone implants can be used, for example, to replace joints, such as in total hip or knee replacement, or to surgically replace bone in the treatment of traumatic injury, bone disease, cancer, or deformity. The implants can contain porous calcium phosphate that could act as a substrate for the drug releasing material. In a preferred embodiment, the coating of drug releasing material on the implant is thin, consisting of as little as one molecular layer of the polypeptide, and readily degradable, so as not to interfere with the primary purpose of the implant, i.e., the eventual replacement of the implant with bone.

The present invention further provides implantable drug releasing materials formed by the methods disclosed herein, as well as dental implants and bone implants comprising the implantable drug releasing materials disclosed herein. In one embodiment of the present invention, the calcium phosphate composition is coated onto an implantable medical device or forms part of an implantable medical device.

The present invention is further directed to methods of delivering a drug to a bone or to a tooth comprising applying the implantable drug releasing materials disclosed herein to the bone or tooth.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Overview

Gentamicin and vancomycin loading and release are presented for a homopolymer system where the antibiotic gentamicin or vancomycin is bound to hydroxyapatite (HA) using commercially available poly-L-glutamate. This corresponds to the schematic illustrated in FIG. 1A. The experiment consisted of producing gentamicin or vancomycin adsorbed on poly-L-glutamate coated hydroxyapatite, and measuring the drug's rate of release into phosphate-buffered saline at 20° C. or 37° C. and pH 7.4, relative to a drug/hydroxyapatite control without the polymer.

### Materials and Methods

Synthesis of hydroxyapatite (HA): The calcium phosphate mineral was precipitated following a method similar to Chen et al. (1984), and characterized by elemental analysis, powder X-ray diffraction (XRD), specific surface area measurements and TEM.

Adsorption of poly(glutamate): The HA was stirred in a solution of poly-L-glutamic acid (Sigma poly-L-glutamic acid, sodium salt, P4886, molecular mass 41,040 by MALLS), filtered, washed, and vacuum dried. The coated product (pgHA) was 4.8% polyglutamate by mass, based on UV analysis of peptide in the filtrate vs. starting solution.

Adsorption of gentamicin: The pgHA powder was stirred in 0.10 mM gentamicin solution, filtered, washed, and vacuum dried. The product (G/pgHA) was 0.83% gentamicin by mass based analysis of gentamicin in the filtrate vs. starting solution. Samples were analyzed by a modified CBQCA fluorescent tag method, using a Molecular Probes Atto-tag kit (A-2333), where the non-fluorescent CBQCA reagent reacts with primary amines on gentamicin to produce a fluorescent product.

Control sample preparation: An HA control sample with an equivalent amount of gentamicin, but no polymer coating (G/HA), was prepared by impregnation of an HA powder sample to incipient wetness with an aqueous gentamicin solution, followed by vacuum drying.

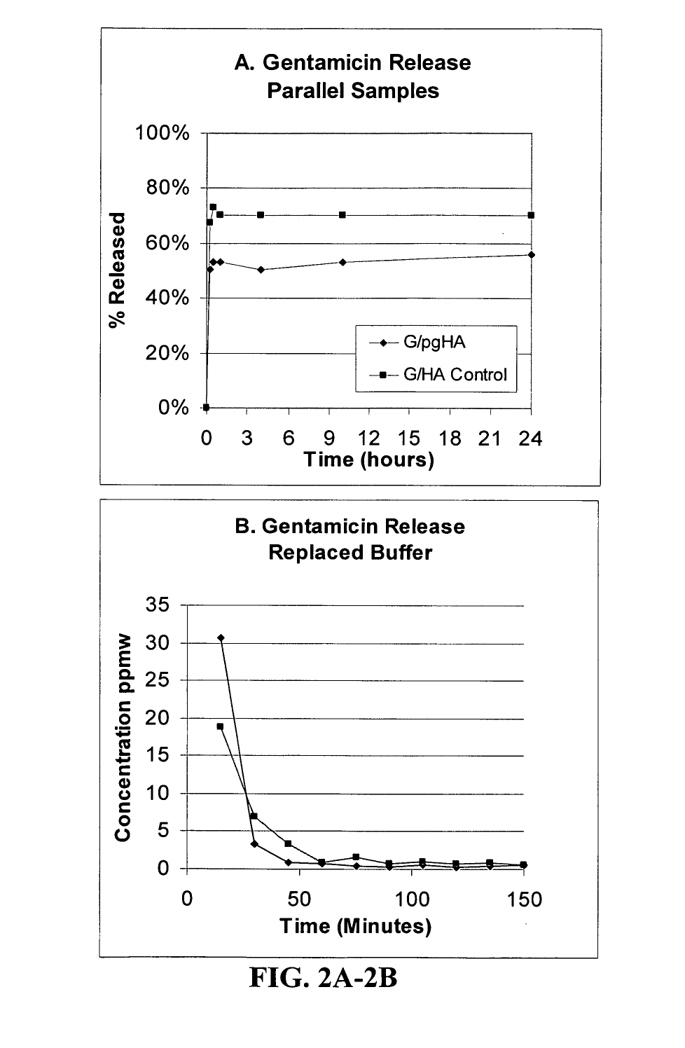

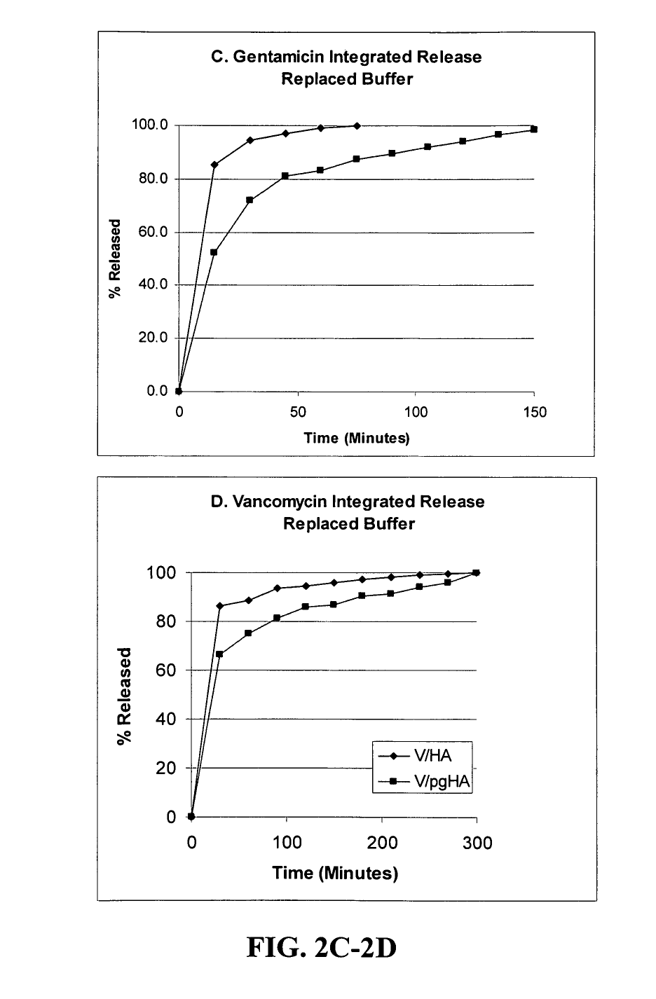

Gentamicin release study—Parallel release reactions: Gentamicin release rates from the experimental sample (G/pgHA) and control (G/HA) were measured by running multiple release reactions of each in parallel and stopping the reactions at different times. In each release sample 6.0 mg of solid was mixed in 1.50 mL of PBS buffer in an Eppendorf tube in a 37° C. shaker bath. Samples were removed from the bath at 15 minute intervals, centrifuged for 30 seconds, and the supernatant saved for later analysis. FIG. 2A shows the percent of gentamicin released into solution for G/pgHA and the G/HA control as a function of time.

Gentamicin release study—Sequential sampling by replacing buffer: In a second release study, gentamicin release rates were measured by mixing 6.0 mg of samples of G/pgHA and G/HA in 1.50 mL of PBS buffer at 37° C. for 15 minutes. The samples were then centrifuged for 30 seconds and the supernatant was removed for later analysis. Fresh buffer was added to each sample and the process was repeated to generate a release profile over time. FIG. 2B shows the supernatant analytical values as a function of time, while FIG. 2C shows the cumulative percent released. Gentamicin release studies were also performed at 20° C., with little difference in results.

Vancomycin release study: These experiments were done very similarly to the gentamicin experiments. The pgHA adsorbed vancomycin to produce a 2.9% vancomycin sample (V/pgHA). A control impregnated with an equivalent amount of vancomycin and dried was made for comparison (V/HA). The adsorption and release experiments were followed by u.v. analysis of vancomycin. FIG. 2D shows the integrated release for V/pgHA vs. the V/HA control, done with sequential sampling by replacing buffer. The experiment was repeated with similar results. Vancomycin release studies were performed at 20° C.

### Results and Discussion

The release profile shown in FIG. 2A shows that adsorbed gentamicin reaches equilibrium with the buffer within one hour. A greater percent of the antibiotic remains adsorbed on the pgHA, however, than on the HA control. This presumably reflects the ionic attraction of the positively charged gentamicin cation at pH 7.4 to the negatively charged, adsorbed polyglutamate.

The release profiles in FIG. 2B and 2C show that gentamicin is released more slowly from G/pgHA than the G/HA control as the buffer is periodically removed and replaced from each sample. This process represents a series of sequential equilibria, analogous to chromatographic migration. The experiment demonstrates that (1) the polymer is adsorbed, (2) that it adsorbs the gentamicin, (3) the extent of release is reduced in the G/pgHA sample relative to the control, (4) the rate of release is slower for G/pgHA under the sequential sampling conditions, and (5) all of the antibiotic is released. Note in FIG. 2C that the control sample released 85% of its gentamicin at the first sampling point (15 minutes), while it took the experimental sample over four times as long to release this amount. Similar results were obtained with the release of vancomycin (FIG. 2D).

The amount of gentamicin loaded onto a small volume of the G/pgHA powder in the experiment is enough to kill bacteria in a medically significant volume of tissue. Calculations based on the weight percent gentamicin in the G/pgHA material show that it contains enough antibiotic per gram of lightly packed powder to kill bacteria in a 500 cc volume of tissue. This is based on a literature value of 4 μg/mL gentamicin as the minimum inhibitory concentration required to kill staphylococci, the most important source of graft infection (de Neeling et al., 1998). The gentamicin loading in the experiment was not maximized. Higher loading may well be achieved by varying loading conditions.

Scaling Effects and In Vitro vs. In Vivo Release Conditions: The data presented herein above used only a few milligrams of product in a small volume of aqueous buffer, where the mineral powder particle size was below 45 microns (<325 mesh). Because of the scale and other factors, these in vitro results show the relatively slowed release from the coated sample, but do not reflect the actual rates of release that would occur from a real implant in a surgical site in vivo. Several factors would make in vivo drug release occur much more slowly. Typically, bone implants made of packed particulates or larger porous objects occupy a volume ranging from one to over a hundred cubic centimeters. As a result, a drug that migrates out into surrounding tissue has to migrate a much longer distance, which would take a longer time. In addition, the current experiment includes active mixing of the fine powder with the buffer, while in an actual surgical site a drug would have to undergo slow diffusion through tissues without mixing. The drug's diffusivity would also be lower, as it typically would diffuse through the semi-solid structure of a hematoma in a surgical site. Thus, in vivo release can be much slower than in vitro test results, with hours in vitro corresponding to days in vivo (Ruszczak et al., 2003). These effects have been investigated where antibiotic loaded implant pieces were surrounded by a layer of clotted blood (Silverman et al., 2007).

The molecularly thin polypeptide layers can eventually be desorbed (Moreno et al., 1984) and biodegrade (Roweton et al., 1997), so as to minimize the potential to interfere with the tissue scaffold function of the implant. The calcium phosphate can be synthesized for this purpose or the polypeptide plus drug layer can be applied to an existing tissue scaffold. In addition to using commercially available linear acidic polypeptides, both branched polypeptides and block copolypeptides can be synthesized for optimized implant surface adsorption and controlled drug release.

Based on the above observations, the present invention exhibits numerous characteristic which provide advantages over the prior art. Specifically, the use of the monolayer provides quick release formulation of the drug, thereby minimizing the potential to breed antibiotic-resistant bacteria. The present invention further avoids particulate formation that can occur upon degradation of thicker polymers, which can lead to inflammation. Furthermore, the use of a monolayer in the present invention minimizes the modification to any surface to which it is bonded (e.g., the surface of a dental or bone implant). Finally, the present invention allows for the application of a high concentration of a drug to the desired site of drug administration, rather than the systemic delivery of the drug.

### REFERENCES

Agrawal C M and Athanasiou K A, Technique to control pH in vicinity of biodegrading PLA-PGA implants, J Biomed Mater Res Appl Biomater 38:105-114 (1997).

Chen C-C, Boskey A L, Rosenberg L C, The inhibitory effect of cartilage proteoglycans on hydroxyapatite growth, Calcified Tissue International 36: 285-90 (1984).

Cordewener F W, Dijkgraff L C, Ong J L, Agrawal C M, Zardeneta G, Milam S B, Schmitz J P, Particulate retrieval of hydrolytically degraded poly(lactide-co-glycolide) polymers, J. Biomed Mater Res 50; 59-66 (2000).

Deming T J, Facile synthesis of block copolypeptides of defined architecture, Nature 390: 386-389 (1997).

de Neeling A J, van Leeuwen W J, Schouls L M, Schot C S, van Veen-Rutgers A, Beunders A J, Buiting A G, Hol C, Ligtvoet E E, Petit P L, S abbe L I, van Griethuysen A J, van Embden J D, Resistance of staphylococci in the Netherlands: surveillance by an electronic network during 1989-1995, Journal of Antimicrobial Chemotherapy 41: 93-101 (1998).

Goldberg H A, Warner K J, Li M C, Hunter G K, Binding of bone sialoprotein, osteopontin and synthetic polypeptides to hydroxyapatite, Connect Tissue Res 42: 25-37 (2001).

Hovis W D, Bucholz R W, Polyglycolide bioabsorbale screws in the treatment of ankle fractures, Foot Ankle Int, 18: 128-31 (1997).

Jo S, Kim J, Kim S W, Reverse thermal gelation of aliphatically modified biodegradable triblock copolymers, Macromol Biosci 6: 923-8 (2006).

Moreno E C, Kresak M, Hay D I, Adsorption of molecules of biological interest onto hydroxyapatite, Calcified Tissue International 36: 48-59 (1984).

Nishiyama N, Yokoyama M, Aoyagi T, Okano T, Sakurai Y, Kataoka K. Preparation and characterization of self-assembled polymer-metal complex micelle from cis-dichlorodiammineplatinum(II) and poly(ethylene glycol)-poly(aspartic acid) block copolymer in an aqueous medium. Langmuir 15: 377-383 (1999).

Qin C, Baba O, Butler W T, Post-translational modifications of SIBLING proteins and their roles in osteogenesis and dentinogenesis, Critical Reviews in Oral Biololgy and Medicine 15: 126-136 (2004).

Roweton S, Huan S J, Swift G, Poly(aspartic acid): synthesis, biodegradation, and current applications, J Environmental Polymer Degradation 5: 175-181 (1997).

Ruszczak Z, Friess W, Collagen as a carrier for on-site delivery of antibacterial drugs, Advanced Drug Delivery Reviews 55: 1679-98 (2003).

Schmidmaier G, Lucke M, Wildemann B, Haas N P, Raschke M, Prophylaxis and treatment of implant-related infections by antibiotic-coated implants: a review, Injury 37 (supplement 2): S105-12 (2006a).

Schmidmaier G, Lucke M, Schwabe P, Raschke M, Haas N P, Wildemann B, Collective review: bioactive implants coated with poly(D,L-lactide) and growth factors IGF-I, TGF-beta 1, or BMP-2 for stimulation of facture healing, J Long Term Eff Med Implants 16: 61-9 (2006b).

Silverman L D, Lukashova L, Herman O T, Lane J M, Boskey A L, Release of gentamicin from a tricalcium phosphate bone implant, Journal of Orthopaedic Research, 25: 23-29 (2007).

Tsortos A, Nancollas G H, The adsorption of polyelectrolytes on hydroxyapatite crystals, J Colloid Interface Sci 209: 109-115 (1999).

Tsortos A, Nancollas G H, The role of polycarboxylic acids in calcium phosphate mineralization, J Colloid Intreface Sci 250: 159-67 (2002).

Vakil R, Kwon G S, Poly(ethylene glycol)-b-poly(epsilon-caprolactone) and PEG-phospholipid form stable mixed micelles in aqueous media, Langmuir 22: 9723-9 (2006).

1. An implantable drug releasing material comprising:
a) a calcium phosphate composition;
b) a biodegradable polymer adsorbed onto the calcium phosphate composition, wherein the polymer comprises acidic amino acid residues; and
c) a drug adsorbed onto or chemically bound to the polymer.

2. The implantable drug releasing material of claim 1, wherein the calcium phosphate composition comprises hydroxyapatite or tricalcium phosphate.

3. The implantable drug releasing material of claim 1, wherein the polymer is a polypeptide polymer.

4. The implantable drug releasing material of claim 1, wherein the polymer comprises residues of aspartic acid and/or glutamic acid.

5. The implantable drug releasing material of claim 1, wherein the polymer comprises phosphoserine.

6. The implantable drug releasing material of claim 1, wherein the polymer is a poly(glutamic acid) polymer or a poly(aspartic acid) polymer.

7. The implantable drug releasing material of claim 1, wherein the polymer comprises branched polypeptides.

8. The implantable drug releasing material of claim 1, wherein the polymer is a block co-polymer.

9. The implantable drug releasing material of claim 8, wherein one block comprises peptide sequences with acidic residues and another block is optimized to bind a drug.

10. The implantable drug releasing material of claim 1, wherein the polymer is bound to the calcium phosphate composition by ionic interaction.

11. The implantable drug releasing material of claim 1, wherein the polymer is formed as a monolayer.

12. The implantable drug releasing material of claim 1, wherein the drug is an antibiotic, a chemotherapeutic agent, an analgesic, a growth factor, an anesthetic, an anti-inflammatory drug or a cell signaling compound.

13. The implantable drug releasing material of claim 1, wherein the drug is an antibiotic.

14. The implantable drug releasing material of claim 13, wherein the antibiotic is an aminoglycoside.

15. The implantable drug releasing material of claim 13, wherein the antibiotic is gentamicin, tobramycin or vancomycin.

16. The implantable drug releasing material of claim 1, wherein the drug is a chemotherapeutic agent.

17. The implantable drug releasing material of any of claim 16, wherein the chemotherapeutic agent is cisplatin.

18. The implantable drug releasing material of claim 1, wherein the ratio of the number of monomers in the polymer to the number of drug molecules is about 10:1.

19. The implantable drug releasing material of claim 1, wherein the drug is bound to the polymer by ionic interaction.

20. The implantable drug releasing material of claim 1, wherein the drug is bound to the polymer by a coordinate bond.

21. A dental implant or a bone implant comprising the implantable drug releasing material of claim 1.

22. (canceled)

23. A method of preparing an implantable drug releasing material comprising:
a) adsorbing a biodegradable polymer to a calcium phosphate composition, wherein the polymer comprises acidic amino acid residues; and
b) adsorbing or chemically binding a drug onto the polymer.

24-47. (canceled)

48. A method of delivering a drug to a bone or to a tooth comprising applying the implantable drug releasing material of claim 1 to the bone or tooth.
